(19) United States
(12) Patent Application Publication
(10) Pub. No.: US 2010/0022961 A1
(43) Pub. Date: Jan. 28, 2010

(54) SUPERTHIN WOUND DRESSING HAVING FOLDED RELEASE SHEET

(75) Inventor: James C. Dewey, New Hempstead, NY (US)

Correspondence Address: BROWN & MICHAELS, PC;400 M & T BANK BUILDING, 118 NORTH TIOGA ST, ITHACA, NY, 14850, US

(73) Assignee: Jentec , Inc., Northvale, NJ (US)

(21) Appl. No.: 12/302,695

(22) PCT Filed: Jun. 21, 2007
(86) PCT No.: PCT/US2007/071736

§ 371 Date: Jul. 15, 2009

Related U.S. Application Data
(60) Provisional application No. 60/816,295, filed on Jun. 23, 2006.

(51) Int. Cl. A61M 25/02; A61F 13/02

(52) U.S. Cl. 604/180; 602/54

(57) ABSTRACT

A wound dressing, which is easy to apply in very few steps, the dressing having a thin film section surrounded by a thicker outside border section. The border section provides rigidity to the dressing by virtue of being thicker than the inner thin film portion of the dressing. The wound dressing has a film on one side of the dressing layer and a release sheet on a skin-contacting side of the dressing layer.

### BACKGROUND OF THE INVENTION

1. Field of the Invention

The present invention relates to the field of dressings that are applied to skin, and more particularly, to the field of dressings having sections for assisting application of the dressing.

2. Related Art

Wound dressings typically comprise a dressing portion, an adhesive, and protective liners. The dressing portion is the part of the wound dressing that makes protective contact with the wound by keeping it clean and by protecting it from exposure to bacteria and other harmful agents in the environment. The adhesive material maintains the dressing portion on the skin. The protective liners protect the dressing portion from bacteria and other contaminants. In hydrocolloid dressings, the hydrocolloid dressing material also has adhesive properties when the hydrocolloid material is mixed with other ingredients, such as a tackifiers, polymeric materials (for example, polyisobutylene and styrene-isoprene block copolymers), and antioxidants as well as additives for a variety of purposes (for example, antimicrobials).

Hydrocolloid dressings have highly absorbent properties. This allows them to be useful even when the dressing has a low profile or thickness. A low-profile, thin film dressing is desirable because the thinness makes the dressing more conformable and easier to wear. Conformability allows the dressing to adapt to the different contours of the human body. A more conformable dressing is more comfortable and maybe used anywhere on the body. Thinner dressings also provide less of an obstacle for clothing or for movement. In the past, extremely thin dressings have been generally limited to medical grade acrylic dressings.

A problem arises as many of these dressings containing an adhesive component are made thinner. Their pliability and adhesiveness make them more difficult to handle and therefore more difficult to apply. Very thin dressings are also more likely to stick to themselves during application becoming virtually useless and ultimately discarded. Dressings should be as easy as possible for a user to apply to a wound site. Users, such as nurses, typically attempt to apply a dressing to a wound as quickly as possible in order to bring a healing-promoting environment to the wound quickly, and to stop the bleeding. From an efficiency standpoint, nurses may have many other tasks to perform, and would further benefit from dressings that require as few steps as possible to apply to the wound. Cost conscious healthcare institutions would prefer to minimize or eliminate the use of dressings that must be discarded because they have stuck to themselves and are difficult or impossible to separate.

Some manufacturers of such thin film dressings have addressed the difficulty of handling thin film dressings by providing a stiffening frame laminate and release papers around the perimeter of the dressing to give the entire dressing more stiffness when being applied in an effort to prevent the dressings from sticking to themselves. This approach has several problems: one, it complicates the manufacture of the dressing; two, the stiffening frame is typically removed and disposed of during application, which actually complicates application; and three, such systems also tend to have a plurality of release liners thereby increasing the number of steps necessary to correctly apply the dressings.

One additional problem associated with these complicated release systems becomes apparent when one considers that the human body essentially has no flat surfaces; it is comprised of many complex curves, i.e. surfaces which curve in more than one direction at the same time. Classic stiff release systems, many containing a plurality of release layers, work well on flat surfaces, but fail to aid in easily applying dressings to the complex curves of the human body in a wrinkle-free (e.g., having no parts folding onto each other or flat with no folds) manner. The failure to reliably aid in applying the dressing in a wrinkle-free manner ultimately compromises the ability of the dressing to protect the wound.

With many wound dressings, the ease of applying a wound dressing depends on the manner in which a surface of the dressing portion is exposed so that it may be applied to the skin. This involves removing one of the protective liners that are applied to the dressing layer during manufacture and packaging. Most wound dressings, however, are made to be sterile until it is applied so that a wound is covered with a sterile environment. Often, in applying a wound dressing, the user may touch the surface of the dressing and possibly contaminate the dressing layer. It would be desirable in wound dressings that the release system or the design of the dressing itself, especially with thin dressings, minimize the possibility of contacting adhesive surface of the dressing.

It would be desirable to have a very thin dressing that may be used on the human skin that is easy to apply without contaminating the dressing during application. Such dressings currently rely on a plurality of release layers one of which, usually the topmost is rigid so as to support the dressings as the other release layers are removed and when the dressing is positioned. It would be very desirable if such a thin flexible film dressing would be so designed such that the dressing itself would support the thin film and provide a dressing that would not stick to itself on application.

### SUMMARY

Systems consistent with the present invention provide wound dressings which are easy to apply in very few steps, especially when the user is wearing surgical or examination gloves, use a minimum of release layers, allow the dressing's sterility to remain uncompromised and allow the positioning of the dressing on the wound to be accomplished in a flat and wrinkle free manner on the complex curves of the human body. Thin film wound dressing consistent with the present invention would be consistent with a design that provides a more rigid outside “border” that would be more rigid by virtue of being thicker than the inner thin film portion of the dressing. The present invention relates to a wound dressing comprising a dressing layer with a skin-contacting surface, which has a first section that is super-thin and a second section substantially surrounding the first section with a thickness greater than the first section. The dressing layer may also have a third section extending from the second section to form an edge of the dressing. The first section, which is super-thin, has a thickness of about 0.02 to about 0.40 mm. The second section, which substantially surrounds the first section to give support, has a thickness greater than the first section and is about 0.50 mm to 1.50 mm. The optional third section of the dressing surrounds the second section to form a peripheral edge section, which has a thickness less than the second section and between about 0.10 and 0.75 mm.

The dressing layer may be made from a water-absorbable material selected from the group consisting of a hydrocolloid, a hydrogel, a foam, polyacrylate, polyvinyl alcohol, and polyvinyl pyrrolidone. If the dressing layer is a hydrocolloid material, it may be calcium carboxymethylcellulose (“CMC”), pectin, gelatin, guar gum, honey, high molecular weight carbowax, carboxypolymethylene.

The dressing may have a film that substantially covers the surface of the dressing layer opposite the skin-contacting surface. The film may be made from polyurethane, polyethylene, polypropylene, styrene-isoprene copolymers, styrene-butadiene block copolymers, butadiene rubbers, isoprene rubbers, neoprene rubbers, acrylonitrile rubbers, silicone rubbers, butyl rubbers, chloroprene rubbers, polyvinylchloride, polyamides, foamed material or non-woven material or mixtures thereof.

The dressing may also have one or more release sheets that cover the skin-contacting surface of the dressing layer to, among other things, maintain the sterility of the skin-contacting surface of the dressing layer. If there is more than one release sheet, it preferably forms a Z-fold that can aid in the application of the dressing. The release sheet is preferably made from a film material having an adhesive resistant surface. The film material may be plastic, PET, metallized plastic, foil or paper.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE FIGURES

The components in the figures are not necessarily to scale, emphasis instead being placed upon illustrating the principles of the invention. In the figures, like reference numerals designate corresponding parts throughout the different views.

FIGS. 1A-1D are perspective views of examples of thin film dressing layers consistent with the present invention.

FIG. 2 is a exploded perspective view of a dressing that incorporates the thin-film dressing layer of FIG. 1A showing the component parts of the dressing separated from one another for purposes of illustration.

FIGS. 3A-3F are different views of one example of the thin film dressing layer with an embossed peripheral section.

FIG. 4A depicts a perspective view of a dressing using an example applicator system consistent with the present invention.

FIG. 4B depicts a cross-sectional view of the dressing of FIG. 4A.

FIG. 4C depicts a cross-sectional view of the dressing of FIG. 4A with the components separated from one another.

FIGS. 5A-5G depicts an example of how the dressing of FIG. 4A may be applied on to a wound bed.

### DETAILED DESCRIPTION

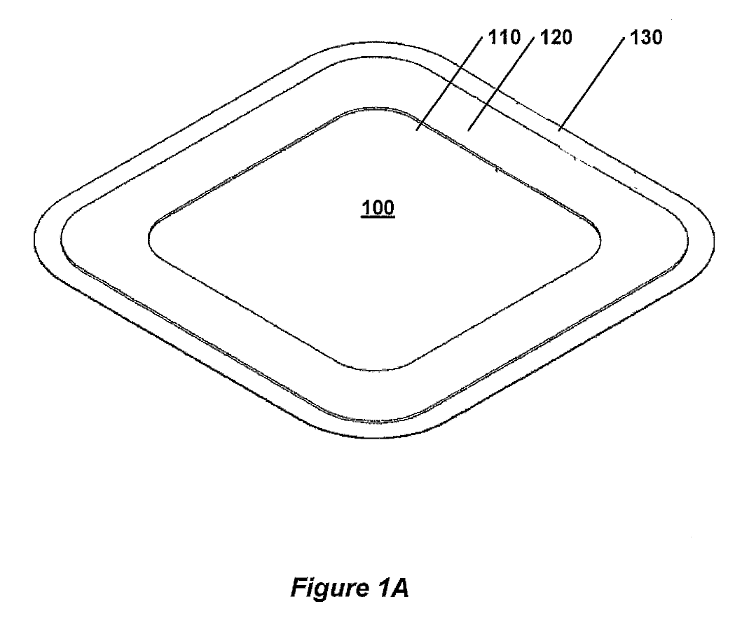

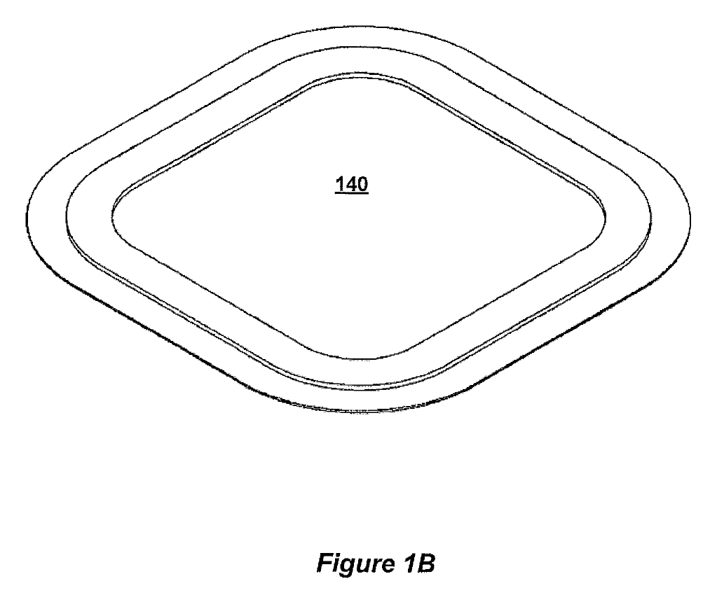

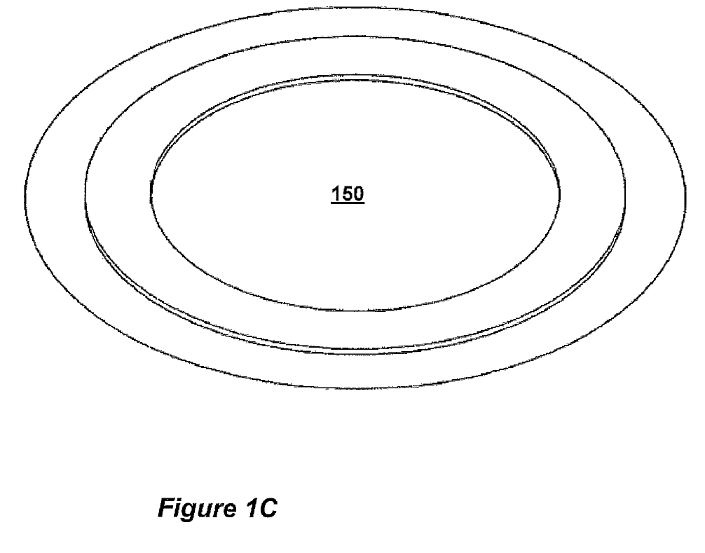

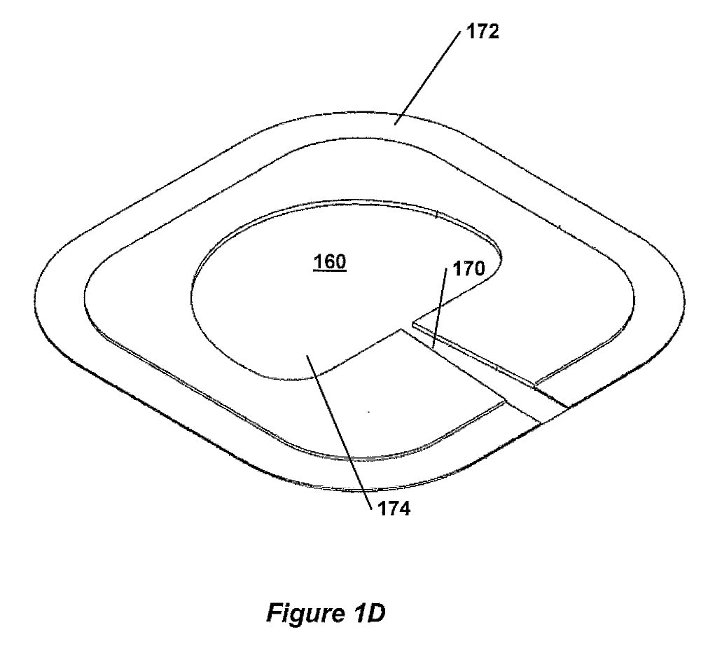

FIGS. 1A-1D are perspective views of examples of thin film dressing layers consistent with the present invention. The differences between the thin film dressing layers shown in FIGS. 1A-1D are primarily differences in shape. The different shapes of the thin film dressing layers of FIGS. 1A-1D illustrate the wide variety of applications served by examples of the present invention. One of ordinary skill in the art will appreciate that the present invention is not limited to any particular shape or configuration.

FIG. 1A shows a perspective view of an example of a substantially rectangular (or square) thin film dressing layer 100 consistent with the present invention. The thin film dressing layer 100 is a hydrocolloid-adhesive film having a first section 110, a second section 120, and a third section 130.

The first section 110 in the example of FIG. 1A is a “superthin” section 110 substantially surrounded by the second section, or raised outer section 120. The “superthin” section 110 is advantageously as thin as it could possibly be made. The term “superthin” is used herein to mean sufficiently thin so as to be difficult to handle without risk of wrinkling in the absence of a stiffening mechanism. In examples of the present invention, the stiffening mechanism that permits simplified handling of the dressing is the raised outer section 120.

The raised outer section 120 is an embossed portion of the dressing layer 100 surrounding the superthin section 110 and having a thickness greater than the thickness of the superthin section 110. The thickness of the raised outer section 120 should be sufficient to impart rigidity to the entire dressing layer 100. This rigidity should be sufficient to prevent the superthin section 110 from folding over itself, or to easily wrinkle.

The raised outer section 120 is surrounded by the third section 130, or edge section 130, that is thinner than the raised outer section 120, but as thin or thicker than the superthin section 110. The edge section 130 advantageously makes the dressing layer 100 more conformable.

The dressing layer 100 in FIG. 1A is substantially rectangular, however, the dressing layer 100 may be any shape. FIG. 1B is a perspective view of a second example of a dressing layer 140 consistent with the present invention in which the dressing layer 140 is substantially rectangular, but has more slightly rounded corners. FIG. 1C is a perspective view of an example of a round dressing layer 150. FIG. 1D is a perspective view of a rectangular dressing layer 160 having a notch 170 in the raised outer section 172. The notch 170 advantageously allows ingress and egress of a medical tube, such as an intravenous catheter tube.

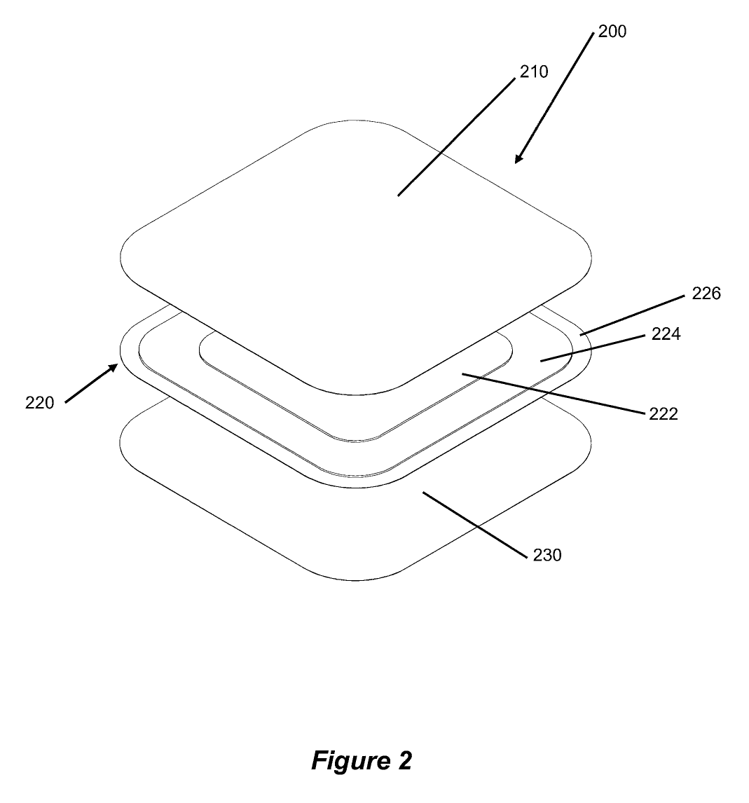

The dressing layer illustrated in FIGS. 1A-1D may be part of a wound dressing system having components that both protect the dressing layer, preferably keeping it sterile, and simplify the process of applying the dressing to a patient. FIG. 2 is an exploded perspective view of an example of a wound dressing system 200 that makes advantageous use of examples of dressing layers consistent with the present invention. The wound dressing system 200 includes a film 210, a dressing layer 220, and a release sheet 230. The release sheet 230 covers the skin-contacting surface of the dressing layer 220. The film 210 remains on the dressing layer 220 on the side opposite the skin-contacting surface of the dressing layer 220.

The release sheet 230 may be one sheet covering the entire skin-contacting surface of the dressing layer 220, or it may be split into one or more sections to provide a gripping surface during application. This gripping surface permits application without the need to touch the skin-contacting surface during application. The release sheet 230 may be made with any material having an adhesive resistant surface on the side of the release sheet 230 that contacts the skin-contacting surface of the dressing layer 220. The first release sheet material should provide flexibility and make the sheet sufficiently rigid to protect the dressing layer portion it covers. In various example embodiments, the release sheet 230 is made of one, or a combination, of plastic, PET (e.g. Mylar™), paper, metallized plastic or foil. The adhesive resistant surface may be a layer of a silicone-based material applied to the film material.

The film 210 may be permanently adhered to the dressing layer 220 to provide a protective layer for the wound dressing 200 as the dressing covers a wound site. The film 210 is preferably made of a transparent material that allows a user to see the wound while the wound dressing 200 covers the wound site. In some example embodiments, the film 210 may be made of polyurethane, polyethylene, polypropylene, or mixtures, or combinations, thereof. The thickness of the film 210 may be extremely thin to prevent imparting any rigidity to the thin-film dressing. Examples of the film 210 may be about 0.03 mm thick.

The dressing layer 220 combines an adhesive material with a material having high moisture absorption properties. The adhesive material secures the dressing layer 220 to the skin at the wound site. The adhesive material may include a tackifier, such as a hydrocarbon resin, and/or acrylic. In one example embodiment, the adhesive material comprises a combination of a tackifier and hot melt acrylic.

The highly moisture-absorbent material may be a hydrocolloid material. One example of a hydrocolloid that may be used is calcium carboxymethylcellulose (“CMC”). Others include pectin, gelatin, guar gum, high molecular weight carbowax, carboxypolymethylene, polyacrylate, polyvinyl alcohol, and polyvinyl pyrrolidone.

In example embodiments, the tackifier is preferably a hydrocarbon resin. The elastomer is preferably a styrene-olefin-styrene polymers, but may also be polyisobutylene, natural rubber, silicone rubber, acrylonitrile rubber, and polyurethane rubber. The dressing layer 16 may also include an extender, preferably paraffin oil. The extender may also be a material that functions as a plasticizer, particularly in combination with the elastomer. Such plasticizers include glycerin (glycerol), sorbitol, triethylene glycol. The extender may also be mineral oil, poly(butene 1) and polyisobutylene.

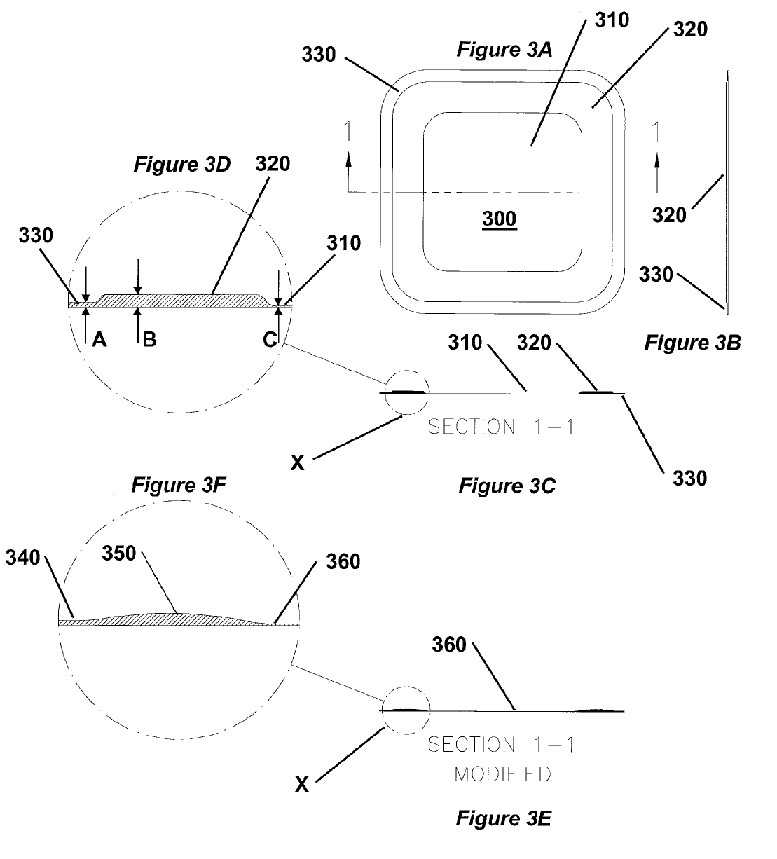

In examples of the wound dressing 200 consistent with the present invention, the dressing layer 220 is “reverse-embossed” in a manner similar to that shown in FIGS. 1A-1D. By “reverse-embossed,” it is meant that the dressing layer 220 includes at least a superthin section 222 and the second section (a raised outer section) 224 (similar to the sections shown in FIG. 1A). The superthin section 222 is substantially thinner than the raised outer section 224. The precise thicknesses of the sections is not critical. The optional third section 226 may be as thin as the superthin section 222, but not as thick as the second section 224. Examples of dimensions for the dressing layer in FIG. 1A are described below with reference to FIG. 3.

FIG. 3A is an overhead view of an example of a dressing layer 300 consistent with the present invention. The dressing layer 300 in FIG. 3A includes a superthin section 310 with a raised outer section 320 and an edge section 330. The outer raised section 320 surrounds the superthin section 310 providing the dressing with rigidity that helps prevent wrinkling and having the dressing fold over itself due to the thinness of the superthin section 310. As discussed in more detail with reference to FIG. 3C below, the superthin section 310 is the thinnest of the three sections. The edge section 330 may be as thin as the superthin section 310. The superthin section 310 is preferably sufficiently thin such that it may tend to wrinkle easily making it difficult to handle when applying on a patient.

One example dressing 300 of the type shown in FIG. 3A may have dimensions of about 200 mm×200 mm. Given those dimensions, the edge section 330 may have a width of about 10 mm. The raised outer section 320 may have a width of about 25 mm. These dimensions leave an area of about 130 mm×130 mm for the superthin section 310. The superthin section 310 preferably has a substantial portion of the area of the dressing 300. In various embodiments consistent with the present invention, the surface area covered by the superthin section 310 may be anywhere from about the same surface area as the raised outer section 320 to up to about 8× the surface area of the raised outer section 320. The raised outer section 320 advantageously provides the entire dressing with stiffness to allow the user to handle the dressing during application. One of ordinary skill in the art will appreciate that the shape and dimensions described herein are merely for purposes of illustration. Neither the size nor shape of any of the examples described herein is intended to limit the scope of the claims.

FIG. 3B is a front view of the dressing 300 viewed from an edge. FIG. 3C is a cross-sectional view of the dressing 300 at section 1-1 of FIG. 3A. FIG. 3D is a cross-sectional view of the dressing in FIG. 3C at Detail X. This view illustrates the relative thicknesses of the edge section 330, the raised outer section 320, and the superthin film section 310. The three sections of the dressing 300 of FIGS. 3A-3D are sections of one dressing layer made of a highly absorbent adhesive material, such as a hydrocolloid adhesive dressing material. The superthin film section 310 may have a thickness, C, of between about 0.02 mm and about 0.40 mm, but preferably about 0.25 mm. The raised outer section 320 may have a thickness, B, of between about 0.50 and about 1.50 mm, and preferably about 1.25 mm. The thickness, A, of the edge section 330 is between about 0.10 and about 0.75 mm, and preferably about 0.40 mm.

FIG. 3D shows the profile of the raised outer section 320 as being a raised flat section relative to the superthin film section 310 and the edge section 330. FIG. 3E is a modified view of the cross-sectional view of the dressing 300 at section 1-1′ illustrating an alternative profile for (with a rounded surface) the raised outer section. FIG. 3F shows this profile as being a rounded surface.

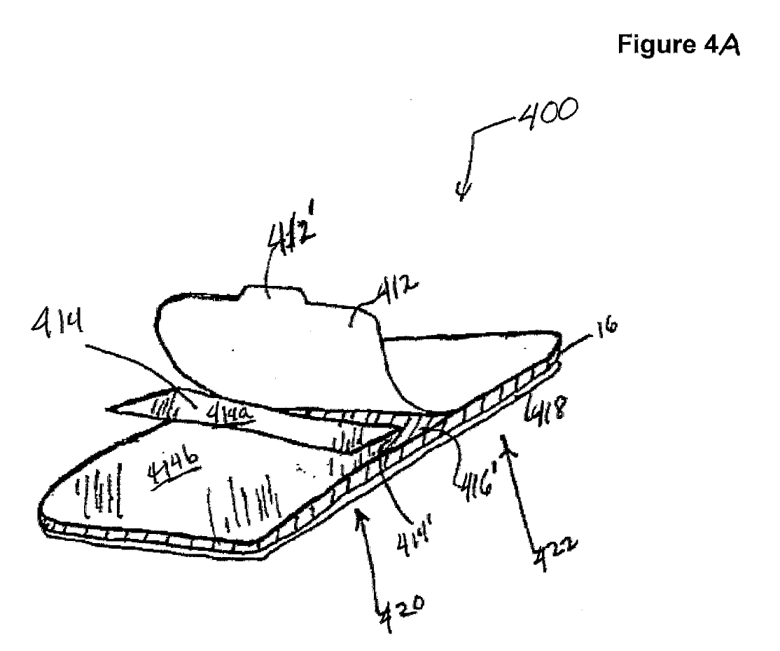

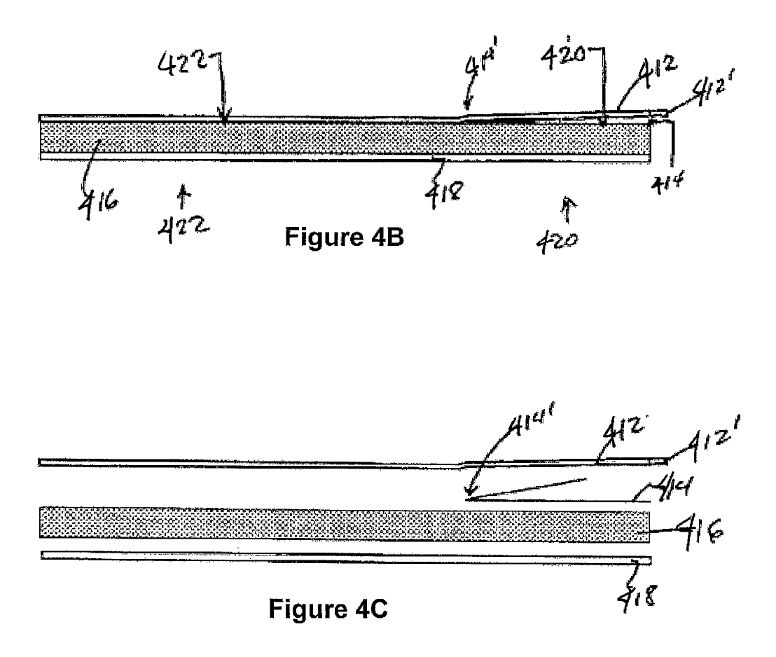

The examples of wound dressings described above with reference to FIGS. 1A-3D provide thin film dressings with thicker sections to provide stiffness for ease of handling during application. The example wound dressings described above may be provided in packaging that provides further ease of application. FIG. 4A shows a perspective view of a wound dressing 400 that uses an example application system consistent with the present invention. The wound dressing 400 comprises a dressing layer 416 covered by a first release sheet 414 and a second release sheet 412 on a skin-contacting surface 416′ of the dressing layer 416. The dressing layer 416 is covered by a backing layer 418 on the side opposite the skin-contacting surface 416′. The dressing layer 416 has a first release sheet region 420 substantially covered by the first release sheet 414 and a second release sheet region 422 substantially covered by the second release sheet 412. The dressing layer 416 may include any example dressing layer described above with reference to FIGS. 1A-3D.

The first release sheet 414 has a fold 414′ that divides the first release sheet 414 into a pull tab area 414a extending from the fold and a dressing cover area covering the first release sheet region 420 of the dressing layer 416. The pull tab area 414a provides a grip that allows a user to pull on the tab to remove the first release sheet 414 from the skin-contacting surface 416′ of the dressing layer 416.

The second release sheet 412 extends over the skin-contacting surface 16′ of the dressing layer 416 on the second release sheet region 422. The area of the second release sheet 412 is generally greater than, and preferably substantially equal to, the area of the second release sheet region 422. The portion of the second release sheet 412 that extends beyond the second release sheet region 422 may be used as a grip to pull on the second release sheet 412 and remove it from the skin-contacting surface 416′. In one example implementation, a grip tab 412′ extends from an edge of the portion of the second release sheet 412 that extends beyond the second release sheet region 422.

The above-described configuration of the wound dressing 400 advantageously simplifies the process of applying the dressing layer 416 to a wound site. A user of the wound dressing 400 may apply the wound dressing 400 by gripping the grip tab 412′ on the second release sheet 412 and pulling on the pull tab area 414′ of the first release sheet 414. The user may then place the exposed portion of the dressing layer 416 to one side of the wound and peel off the second release sheet 412 while covering the wound with the remainder of the dressing layer 416 as it becomes exposed. Application of the wound dressing 400 may therefore be accomplished without requiring the user to touch the skin-contacting surface 416′ in an easy, efficient manner.

FIG. 4B depicts a cross-sectional view of the wound dressing 400 in FIG. 4A in accordance with an example consistent with the present invention. As shown in FIG. 4B, the wound dressing 400 is preferably flat to allow for packaging in an envelope (not shown) that may be sealed to protect the wound dressing 400 from outside contaminants. The wound dressing 400 is preferably assembled and packaged in a sterile environment, such as a clean room. The sealed envelope keeps the wound dressing 400 sterile until the user applies the wound dressing 400 to a wound site. The configuration of the wound dressing 400 allows the user to apply the dressing 400 without touching the skin-contacting surface 416′ thereby allowing the dressing 400 to remain sterile as it is applied.

Referring to FIG. 4B, the second release sheet 412 substantially covers the area of the dressing layer 416 and makes contact with the skin-contacting surface 416′ in the second release sheet region 422. The first release sheet 414 covers the dressing layer and makes contact with the skin-contacting surface 416′ in the first release sheet region 420. The fold 414′ in the first release sheet 414 serves as a boundary that divides the first release sheet region 420 from the second release sheet region 422.

FIG. 4C is a cross-sectional view of the wound dressing 400 with its component layers separated. FIG. 4C illustrates more clearly how the components fit together to form the wound dressing 400.

The first release sheet 414 is preferably made of a film material having an adhesive resistant surface on the dressing cover area, which is the portion that contacts the dressing layer 416. The first release sheet material should provide flexibility and make the sheet sufficiently rigid to protect the dressing layer portion it covers. In example embodiments, the first release sheet 414 is made of one, or a combination, of plastic, mylar, PET, and paper. The adhesive resistant surface may be a layer of a silicone-based material applied to the film material.

The second release sheet 412 is preferably made of a protective material having an adhesive resistant surface on the side covering the second release sheet region 422 of the skin-contacting surface 416′ of the dressing layer 416. The first release sheet material should provide flexibility and make the sheet sufficiently rigid to protect the dressing layer portion it covers. The protective material may be a material selected from the group consisting of paper, cardboard, PET, plastic, and mylar. The adhesive resistant surface may be a layer of a silicone-based material applied to the film material. In an example implementation, the second release sheet 412 is made of siliconized paper.

The backing layer 418 may be permanently adhered to the dressing layer 416 thereby providing a protective layer for the wound dressing 400 as the dressing covers a wound site. The backing layer 418 is preferably made of a transparent material that allows a user to see the wound while the wound dressing 400 covers the wound site. Alternatively, the backing layer 418 may be an adhesive resistant material that may be removed upon application to the wound site. In example embodiments, the backing layer 418 may be made of polyurethane, polyethylene, polypropylene, or mixtures thereof.

The dressing layer 416 preferably combines an adhesive material with a material having high moisture absorption properties. The adhesive material secures the dressing layer 416 to the skin at the wound site. The adhesive material may include a tackifier, such as a hydrocarbon resin. In one example embodiment, the adhesive material comprises a combination of a tackifier and hot melt acrylic.

The highly moisture-absorbent material may be a hydrocolloid material. One example of a hydrocolloid that may be used is calcium carboxymethylcellulose (“CMC”). Others include pectin, gelatin, high molecular weight carbowax, carboxypolymethylene, polyacrylate, polyvinyl alcohol, and polyvinyl pyrrolidone.

In example embodiments, the tackifier is preferably a hydrocarbon resin. The elastomer is preferably a styrene-olefin-styrene compound, but may also be polyisobutylene, natural rubber, silicone rubber, acrylonitrile rubber, and polyurethane rubber. The dressing layer 416 may also include an extender, preferably paraffin oil. The extender may also be a material that functions as a plasticizer, particularly in combination with the elastomer. Such plasticizers include glycerin (glycerol), sorbitol, triethylene glycol. The extender may also be mineral oil.

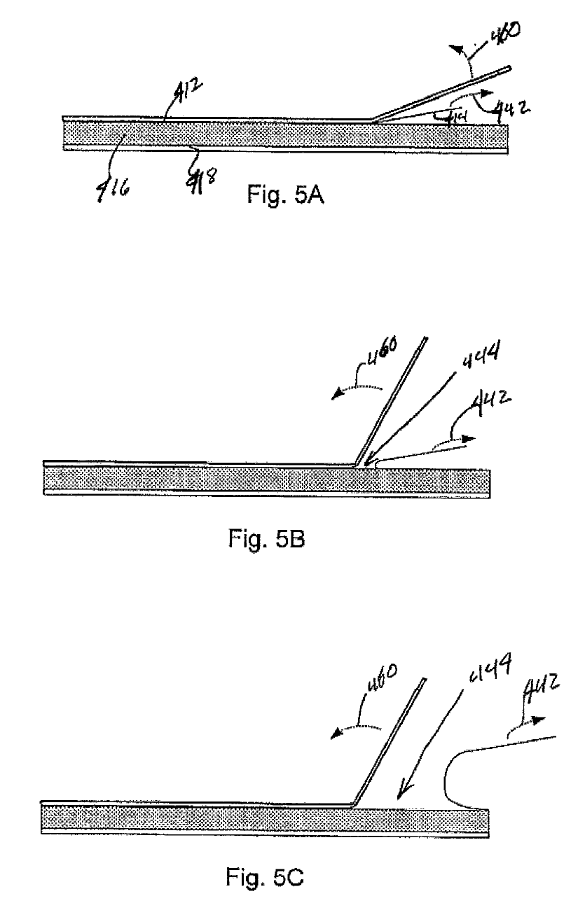

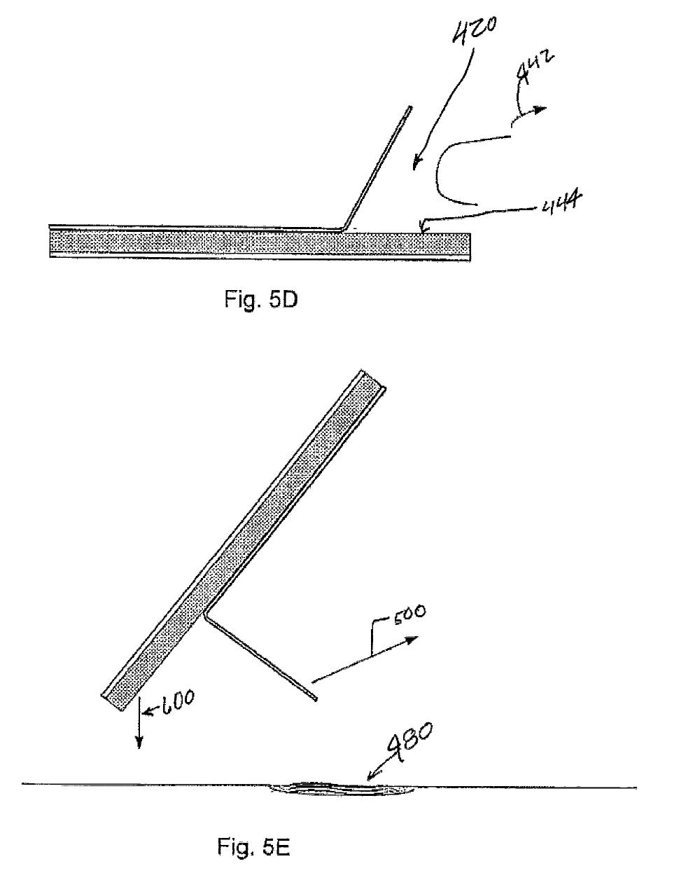

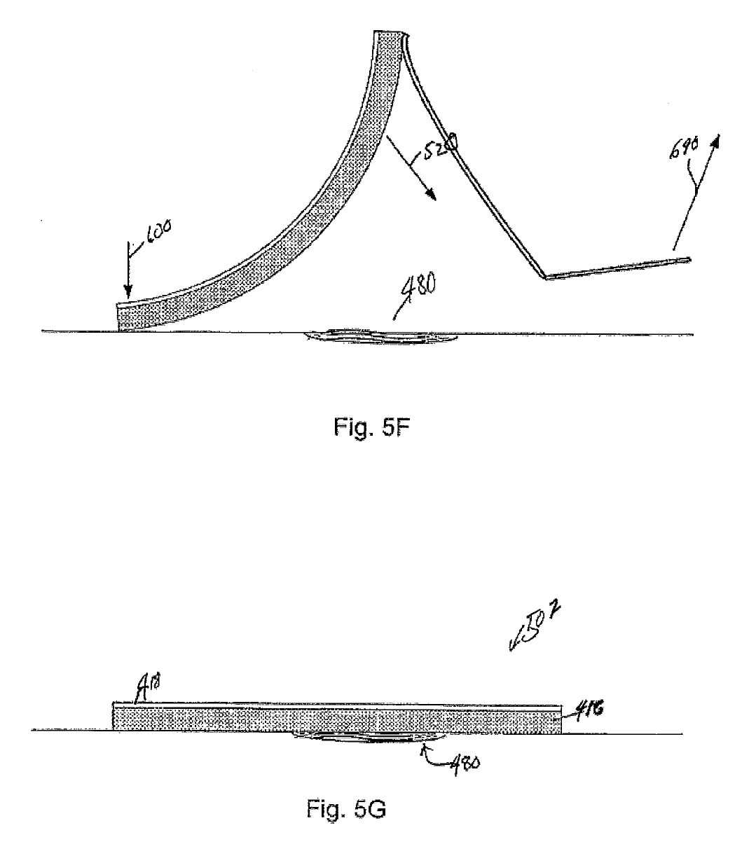

FIGS. 5A-5G illustrate how the wound dressing 400 may be applied to a wound site. As shown in FIG. 5A, a user separates the second release sheet 412 from the area of the first release sheet 414 by pulling on the second release sheet 412 in the direction shown by arrow 460. The user may then grip the first release sheet 414 and pull in the direction shown by arrow 442 to begin removing the first release sheet 414.

FIG. 5B depicts the manner in which an exposed area 444 on the dressing layer 416 begins to emerge from the pulling of the first release sheet 414 in the direction shown by arrow 442 and the pulling of the second release sheet 412 in the direction shown by arrow 460. FIG. 5C shows an even larger exposed area 444 as the first release sheet 414 continues to come off in the direction shown by arrow 442. FIG. 5D shows the completely exposed area 444 of the dressing layer 416 in the first release sheet region 420 after the complete removal of the first release sheet 414.

With the first release sheet 414 completely removed, the user may turn the wound dressing 400 upside down and begin to apply the wound dressing 400 as shown in FIG. 5E. An exposed part of the dressing layer is pressed down on the skin as shown by the arrow 600. The user may then peel off the second release sheet 412 by pulling in the direction shown by the arrow 500 exposing more of the dressing layer. Referring to FIG. 5F, as more of the dressing layer is exposed, the dressing layer is moved in the direction shown by arrow 520 towards a wound site 480. When the second release sheet 412 is completely removed, the dressing layer 416 may then fully rest on the wound site 480 as shown in FIG. 5G. The wound dressing 400 is now resting on the wound site 480 and the dressing layer 416 is covering the wound site 480 providing it with moisture absorbing capabilities. Moreover, because the dressing 400 was applied without the need for the user to touch the skin-contacting surface 416′ of the dressing layer 416, the dressing 400 is still substantially sterile providing a clean environment that promotes healing at the wound site.

Example embodiments of the present invention have been described. Persons of ordinary skill in the art will appreciate that variations may be made without departure from the scope and spirit of the invention. For example, one of ordinary skill in the art will appreciate that the wound dressing 400 may be of any shape or size. This true scope and spirit is defined by the appended claims, interpreted in light of the foregoing.

1. A wound dressing comprising:
an adhesive dressing layer having a skin-contacting surface, wherein the dressing layer has a first section having a thickness of about 0.02 to about 0.40 mm, and a second section substantially surrounding the first section and having a thickness greater than the first section.

2. The dressing of claim 1 further comprising a third section extending from the second section to form an edge of the dressing.

3. The dressing of claim 1 comprising:
a film substantially covering a surface of the dressing layer opposite the skin-contacting surface.

4. The dressing of claim 1 further comprising;
a release sheet having an adhesive resistant surface covering the skin-contacting surface of the dressing layer.

5. The dressing of claim 1 wherein the dressing layer further comprises a water-absorbable material selected from the group consisting of a hydrocolloid, a hydrogel, a foam, polyacrylate, polyvinyl alcohol, and polyvinyl pyrrolidone.

6. The dressing of claim 1 wherein the first section is surrounded by the second section and the second section is made of a moisture absorbable material of substantially greater thickness than the first section.

7. The dressing of claim 5 wherein the dressing layer contains a hydrocolloid material selected from the group consisting of calcium carboxymethylcellulose (“CMC”), pectin, gelatin, guar gum, honey, high molecular weight carbowax, and carboxypolymethylene.

8. The dressing of claim 3 wherein the film is made of a material selected from the group consisting of polyurethane, polyethylene, polypropylene, styrene-isoprene copolymers, styrene-butadiene block copolymers, butadiene rubbers, isoprene rubbers, neoprene rubbers, acrylonitrile rubbers, silicone rubbers, butyl rubbers, chloroprene rubbers, polyvinylchloride, polyamides, foamed material and non-woven material, or mixtures thereof.

9. The dressing of claim 4 wherein the release sheet is made of a material having an adhesive resistant surface on the dressing cover area and the film material selected from the group consisting of a plastic, PET, metallized plastic, foil and paper.

10. The dressing of claim 4 wherein the adhesive resistant surface is silicone-based.

11. The dressing of claim 1 wherein the second section completely surrounds the first section.

12. The dressing of claim 1 wherein the second section substantially surrounds the first section to form a gap in the second section where the thickness of the dressing within the gap is the same as the thickness of the first section.

13. The dressing of claim 1 further comprising a third section surrounding the second section to form a peripheral edge section, the third section having a thickness less than the second section.

14. A wound dressing comprising:
a dressing layer having a skin-contacting surface and a moisture absorbent, adhesive composition, the dressing layer having a first section having a thickness of about 0.02 to about 0.40 mm, and a second section substantially surrounding the first section and having a thickness greater than the first section and between about 0.50 mm and 1.50 mm.

15. The dressing of claim 14 further comprising a third section surrounding the second section to form a peripheral edge section, the third section having a thickness less than the second section and between about 0.10 and 0.75 mm.

16. The dressing of claim 14 wherein the second section substantially surrounds the first section to form a gap in the second section where the thickness of the dressing within the gap is the same as the thickness of the first section.

17. The dressing of claim 14 wherein the second section completely surrounds the first section.

18. A wound dressing comprising:
a dressing layer having a skin-contacting surface and an adhesive composition, the skin-contacting surface having a first release sheet region and a second release sheet region, the dressing layer further including a superthin section, and a surrounding section substantially surrounding the superthin section and having a thickness greater than the superthin section;
a backing layer substantially covering a surface of the dressing layer opposite the skin-contacting surface;
a first release sheet having a fold that divides the first release sheet into a pull tab area extending from the fold and a dressing cover area covering the first release sheet region such that the fold defines a boundary between the first release sheet region and the second release sheet region;
and a second release sheet having an area greater than the second release sheet region, the second release sheet covering the skin-contacting surface on the second release sheet region.

19. The dressing of claim 18 wherein the dressing layer further comprises a water-absorbable material selected from the group consisting of a hydrocolloid, a hydrogel, a foam, polyacrylate, polyvinyl alcohol, and polyvinyl pyrrolidone.

20. The dressing of claim 18 wherein the dressing layer contains a hydrocolloid material selected from the group consisting of: calcium carboxymethylcellulose (“CMC”), pectin, gelatin, high molecular weight carbowax, carboxypolymethylene.

21. The dressing of claim 18 wherein the backing layer is made of a material selected from the group consisting of polyurethane, polyethylene, and polypropylene, or mixtures thereof.

22. The dressing of claim 18 wherein the first release is made of a film material having an adhesive resistant surface on the dressing cover area, the film material selected from the group consisting of a plastic, a mylar, PET and paper.

23. The dressing of claim 22 wherein the adhesive resistant surface comprises a silicone-based layer.

24. The dressing of claim 18 wherein the second release sheet is made of a protective material having an adhesive resistant surface on the side covering the second release sheet region of the skin-contacting surface of the dressing layer, the protective material selected from the group consisting of paper, cardboard, PET, mylar and plastic.

25. The dressing of claim 18 wherein the second release sheet has an area substantially equal to the area of the first release sheet region and the second release sheet region.

26. The dressing of claim 18 wherein the second release sheet comprises a grip tab extending from an edge of the second release sheet opposite the portion of the second release sheet that covers the second release sheet region.

27. The dressing of claim 25 wherein the adhesive resistant surface comprises a silicone-based layer.

28. A method of using the dressing of claim 1 to affix a medical tube to a patient in need thereof.

29. The method of claim 28 wherein the medical tube is a gastronomy tube, esophostomy tube, PIC line, or intravenous catheter.

30. The dressing of claim 2 further comprising: a film substantially covering a surface of the dressing layer opposite the skin contacting surface.

31. The dressing of claim 2 further comprising: a release sheet having an adhesive resistant surface covering the skin-contacting surface of the dressing layer.

32. The dressing of claim 30 wherein the film is made of a material selected from the group consisting of polyurethane, polyethylene, polypropylene, styrene-isoprene copolymers, styrene-butadiene block copolymers, butadiene rubbers, isoprene rubbers, neoprene rubbers, acrylonitrile rubbers, silicone rubbers, butyl rubbers, chloroprene rubbers, polyvinylchloride, polyamides, foamed material and non-woven material, or mixtures thereof.

33. The dressing of claim 32 wherein the release sheet is made of a material having an adhesive resistant surface on the dressing cover area and the film material selected from the group consisting of a plastic, PET, metallized plastic, foil and paper.

34. A method of using the dressing of claim 18 to affix a medical tube to a patient in need thereof.

35. The method of claim 34 wherein the medical tube is a gastronomy tube, esophostomy tube, PIC line, or intravenous catheter.
